(19) United States
(12) Patent Application Publication
(10) Pub. No.: US 2006/0004526 A1
(43) Pub. Date: Jan. 5, 2006

(54) System and method for processing results of a complex genetic test

(75) Inventors: Andrew G. Hadd, Austin, TX (US); Cindy R. WalkerPeach, Austin, TX (US); Christopher J. Neil, Cedar Park, TX (US)

Correspondence Address: BAKER BOTTS L.L.P., 2001 ROSS AVENUE, SUITE 600, DALLAS, TX, 75201-2980, US

(73) Assignee: Ambion, Inc.

(21) Appl. No.: 10/883,186

(22) Filed: Jul. 1, 2004

(51) Int. Cl. C12Q 1/68; G06F 19/00; G01N 33/48; G01N 33/50

(52) U.S. Cl. 702/20; 435/6

(57) ABSTRACT

A system and method for processing results of a complex genetic test are disclosed. The method includes receiving raw data from a genetic analyzer that performs a genetic test on a plurality of patient samples including at least one control bead for providing at least one fluorescent control signal contained in a plurality of wells on a plate. The results are calculated for the genetic test by applying a signal intensity associated with the fluorescent control signal to the raw data and a user interface is generated to display the results associated with each of the patient samples.

### TECHNICAL FIELD OF THE INVENTION

The present invention relates in general to genetic testing and, more particularly to a system and method for processing results of a complex genetic test.

### BACKGROUND OF THE INVENTION

The rapid growth in scope and volume of molecular testing for inherited single-gene disorders has engendered both great excitement and significant concern. With the potential targets for testing, particularly in autosomal recessive diseases, including vast numbers of healthy people in high-risk ethnic groups or even the whole population, a strong financial incentive has added further impetus to the rapid translation of research results to clinical testing. The lack of sufficient attention to issues of clinical utility, test validation, and quality assurance in the clinical tests, however, has raised concerns that the transition from gene discovery to diagnostic reagent may be moving ahead too quickly.

The increased volume of genetic testing has also raised concerns about how to effectively manage and properly interpret the collected results and how to provide the patient with the most meaningful information. One test that is currently being performed more frequently is the test for DNA mutations indicative of cystic fibrosis (CF). A conventional CF assay may test a patient sample for the presence of multiple mutations (e.g., in some cases more than twenty-five mutations) that are known to cause CF. Managing the results for the multiple mutations over multiple patient samples has quickly become an issue for clinical labs running the tests. As complex genetic tests become a routine aspect of healthcare, systems that cannot properly obtain and manage results of these tests may suffer from disadvantages that could slow advances in the genetic testing area.

While numerous advances have been made to improve the quality and reliability of the results, conventional techniques for performing and managing the results of a complex genetic test, such as a CF assay, may suffer from numerous disadvantages. For example, in order to increase throughput, a conventional CF assay may be used to test each DNA sample for multiple mutations that are known to cause CF. The results may be pooled and therefore, may only indicate that the patient is a carrier of CF without identifying the specific mutation that causes CF.

Another area of concern is the combination of primary mutations and reflex polymorphisms in a single test. Within CF, reflex polymorphisms such as 5/7/9T in Intron 8 are associated with CF only when in the presence of a primary mutation (e.g., R117H). Without the primary mutation, 5T allele detection is associated with another genetic disease. Detection of the severity without the presence of an associated core mutation may cause, for example, a pregnant woman to undergo risky diagnostic tests even though there is no possibility that the child will either be a carrier of a CF mutation or that the child may be born with CF. This example may become more common as complex genetic disease testing covers a wide range of possible outcomes where the exact severity of certain mutations are unknown. Without the ability to obtain clinically relevant data and manage the acquisition of non-disease results, these types of false detections will continue to occur.

Conventional techniques for performing genetic tests also may not allow a laboratory director to have access to raw data or processing parameters from the test that is used to make an allele call. The director may only be able to view the processed results and may not be able to see how the raw data was manipulated or to affect the parameters used in making an allele call. With limited access to either the raw data or processing parameters, variation in results, performance metrics, and quality of data interpretation may be affected. Furthermore, as tests change or the criteria necessary for assigning mutation changes, the analysis software should provide access to all aspects of the data and processing parameters, and enable results trending.

Another aspect of high-throughput genetic testing is the requirement of control samples for tracking laboratory performance and for use in calculating results in patient samples. Typically, control samples are placed with a batch of patient samples and occupy a reaction well within that batch. In a conventional technique, one of the wells on the plate may be used as a reference well and the genetic analyzer may calculate the results for the other wells based on the measurements obtained from the reference well. Use of a reference well, therefore, may limit the number of samples that may be tested at one time and may cause consistency problems in the well-to-well results due to the lack of internal controls in each well.

A further disadvantage associated with conventional techniques for reporting the results of a genetic test is that a lab director or other clinician must qualitatively or manually interpret the results. For example, the results may be presented in a manner in which the clinician has to manually review paper strips and qualitatively assign an allele call. Trending differences in batches of samples over time by manually reviewing the results, however, may be very difficult. In the case where the results are presented in electronic format, the data may be available only in a raw signal intensity, which requires an additional layer of analysis to calculate the parameters for an allele call. Finally, conventional test systems do not allow network access to the data tracking and patient sample analysis.

Additionally, complex genetic testing requires the ability to adapt to rapidly changing recommendations in the number of genetic tests, mutations within those tests, and reporting guidelines. Conventional techniques typically require substantial changes in the acquisition technique or software to accommodate the changing recommendations. As genetic testing expands into pharmacogenomics and more routine DNA analysis, the ability of a testing system to be easily adaptable will be crucial.

### SUMMARY OF THE INVENTION

In accordance with teachings of the present invention, disadvantages and problems associated with processing results a complex genetic test have been substantially reduced or eliminated. In a particular embodiment, a signal intensity associated with at least one fluorescent control signal in each patient sample is applied to raw data collected for a genetic test to calculate the results of the genetic test.

In accordance with one embodiment of the present invention, a method for processing results of a complex genetic test includes receiving raw data from a genetic analyzer that performs a genetic test on a plurality of patient samples including at least one control bead for providing at least one fluorescent control signal contained in a plurality of wells on a plate. The results are calculated for the genetic test by applying a signal intensity associated with the fluorescent control signal to the raw data and a user interface is generated to display the results associated with each of the patient samples.

In accordance with another embodiment of the present invention, a system for processing results of a complex genetic test includes a genetic analyzer and a computer system. The genetic analyzer performs a genetic test on a plurality of patient samples including at least one control bead for providing at least one fluorescent control signal contained in a plurality of wells on a plate and collects raw data for the genetic test. The computer system includes a processor, a computer readable memory operably coupled to the processor, and processing instructions encoded in the computer readable memory. When executed by the processor, the processing instructions calculate results of the genetic test by applying a signal intensity associated with the fluorescent control signal to the raw data and generate a user interface to display the results associated with each of the patient samples.

In accordance with a further embodiment of the present invention, a method for processing results of a complex genetic test includes receiving raw data from a genetic analyzer that performs a genetic test on a plurality of patient samples contained in a plurality of wells and calculating results of the genetic test based on the raw data. A user interface is generated that displays at least one of a summary chart including the raw data and the calculated results and a patient report associated with the calculated results for each of the patient samples.

In accordance with an additional embodiment of the present invention, a graphical user interface for displaying results of a complex genetic test on a display device includes a summary chart and a patient report associated with each of a plurality of patient samples. The summary chart contains at least one of raw data associated with a plurality of patient samples collected by a genetic analyzer that performs a genetic test, background corrected data calculated based on the raw data corresponding to each of the patient samples, an allele ratio calculated based on the background corrected data corresponding to each of the patient samples and an allele call determined based on the allele ratio corresponding to each of the patient samples.

Important technical advantages of certain embodiments of the present invention include a genetic testing technique that provides an internal control signal in each of the patient samples being tested. In some CF assays, color-coded microspheres, also referred to as beads, may be used to detect mutations on certain alleles. Additional beads, referred to as control beads, may contain oligos that lack homology to any human, mouse or rat and may be used to provide reference signal intensities in each well of the microtiter plate. These reference signal intensities may be used to validate the results of a CF assay and a reflex test, to calculate background corrected values from the raw data collected by the genetic analyzer, and to diagnose problems with the tests and/or lab equipment performing the tests.

Another important technical advantage of certain embodiments of the present invention includes a processing module that electronically links results of a genetic test and a corresponding reflex test. When a genetic test and a reflex test are performed simultaneously, the results of the reflex test may identify severity mutations without the presence of a mutation on the corresponding reflex allele. If the reflex test results are analyzed without determining if the corresponding reflex allele has a mutation, a false positive result may be reported. By either separately performing a reflex test only if a mutation is detected on a reflex allele in the genetic test or by ignoring the results of the reflex test unless the genetic test identifies a mutation on a reflex allele, false positive results may be eliminated.

A further important technical advantage of certain embodiments of the present invention includes a report module that generates a graphical user interface (GUI) to display raw data collected by a genetic analyzer and calculated results of a genetic test. The raw data for a genetic test and/or a reflex test may be compiled in a digital output file by a genetic analyzer. A computer system interfaced with the genetic analyzer may receive the file and calculate the results of the genetic test and/or reflex test. By providing the raw data in the GUI, a user may analyze the raw data to determine whether the genetic analyzer performed the test correctly and/or whether the operator at the genetic analyzer performed his or her job correctly.

An additional important technical advantage of certain embodiments of the present invention includes a report module that generates a color-coded plate view for the results of a genetic and/or reflex test. The plate view includes a graphical representation of each well in a microtiter plate. The graphical representation indicates whether the results of the test performed were normal, a genetic mutation was found, the results were indeterminate, and/or the test failed, which allows a user to quickly review the results of the test for all patient samples tested on a well-by-well basis.

An additional important technical advantage of certain embodiments of the present invention includes a report module that provides a pattern recognition feature that allows a user to visually interpret the results of a genetic test and/or a reflex test. The report module applies scaling factors to background corrected data to generate a virtual line blot representing the results of the genetic test and/or the reflex test. By providing a graphical representation of the results, the report module facilitates management of large data sets such that a user may efficiently interpret the results.

Another important technical advantage of certain embodiments of the present invention includes a report module that provides the ability to filter the results of the genetic test. The results for each patient sample tested may be displayed on a GUI generated by a computer system. A user of the computer system may select the patient sample results to display, for example, in a patient report or a virtual line blot by using a filter tool to select only the results associated with an allele call. By filtering the results based on the allele call, a user may insert a standard comment in a patient report for each of the patient samples having the selected allele call.

All, some, or none of these technical advantages may be present in various embodiments of the present invention. Other technical advantages will be readily apparent to one skilled in the art from the following figures, descriptions, and claims.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1 illustrates a block diagram of a system for processing results of a complex genetic test in accordance with teachings of the present invention;

FIG. 2 illustrates a block diagram of a computer system for processing results of a complex genetic test in accordance with teachings of the present invention;

FIG. 3 illustrates a flow chart of a method for processing results of a complex genetic test in accordance with teachings of the present invention;

FIG. 4 illustrates a flow chart of a method for validating control beads used in a complex genetic test in accordance with teachings of the present invention;

FIG. 5 illustrates one example embodiment of a graphical user interface (GUI) displaying results of a complex genetic test in accordance with teachings of the present invention;

FIG. 6 illustrates another example embodiment of a GUI displaying a comments field associated with a patient report in accordance with teachings of the present invention; and

FIGS. 7A and 7B illustrate an additional example embodiment of a GUI displaying a virtual line blot that provides a visual representation of results of a complex genetic test in accordance with teachings of the present invention.

### DETAILED DESCRIPTION OF THE INVENTION

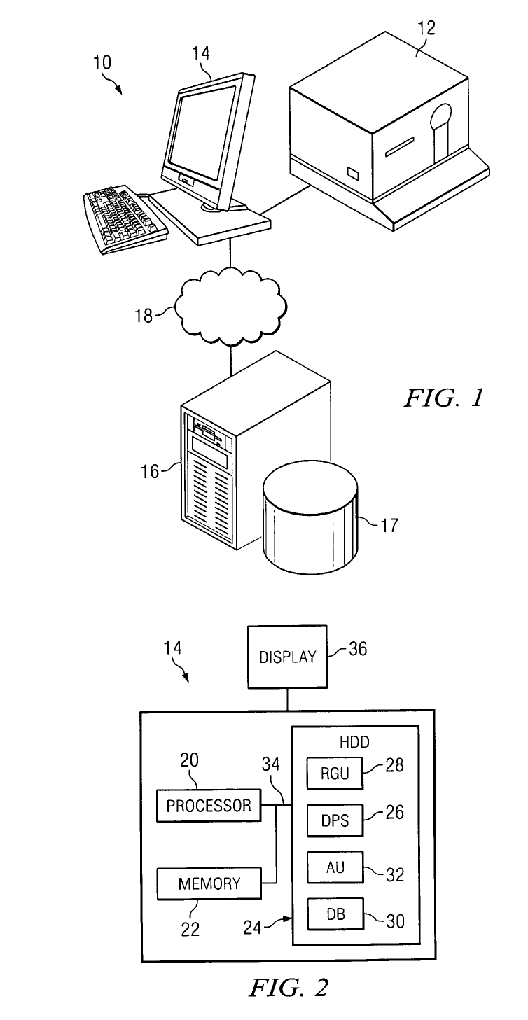

Preferred embodiments of the present invention and their advantages are best understood by reference to FIGS. 1 through 7, where like numbers are used to indicate like and corresponding parts.

FIG. 1 illustrates system 10 for performing and processing results of a complex genetic test. In the illustrated embodiment, system 10 includes genetic analyzer 12, computer system 14 and server 16 interfaced together by network 18. In one embodiment, genetic analyzer 12 performs a genetic test on DNA samples contained in a plurality of wells on a microtiter plate (not expressly shown) and collects raw data for each of the samples to include in a digital output file. Computer system 14 receives the output file from genetic analyzer 12 and calculates the results of the genetic test for each of the samples by applying a signal intensity associated with a fluorescent signal generated by at least one control bead in each of the samples. Computer system 14 then generates a graphical user interface (GUI) including the raw data and the calculated results that allows a lab director, medical director or other doctor to make determinations based on the results and add comments to a patient report associated with each one of the samples tested.

Although a specific communication network is illustrated in FIG. 1, the term “network” should be interpreted as generically defining any network capable of transmitting telecommunication signals, data and/or messages. Network 18 represents any suitable collection and arrangement of communications equipment supporting the transport and delivery of signals, packets, cells, or other portions of information (generally referred to as signals). For example, network 18 may be one or a collection of components associated with the public switched telephone network (PSTN), a local area network (LAN), a wide area network (WAN), a global computer network such as the Internet, or any other communications equipment suitable for providing wireless and/or wireline communications. In operation, network 18 routes various signals including information associated with communication sessions along different physical paths.

System 10 includes genetic analyzer 12 that performs a genetic test on multiple DNA samples, also referred to as patient samples, from different patients. Generally, genetic analyzer 12 may simultaneously assay multiple analytes in a single well of a microtiter plate by using a small sample of blood from a patient. The assays may include, but are not limited to, nucleic acid assays, receptor-ligand assays, immunoassays and enzymatic assays. In one embodiment, genetic analyzer 12 may be a LUMINEX® system manufactured and sold by Luminex Corporation.

Computer system 14 may be any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, computer system 14 may be a personal computer, a portable computer, a workstation, a server, or any other suitable device and may vary in size, shape, performance, functionality, and price. Computer system 14 may include random access memory (RAM), one or more processing resources such as a central processing unit (CPU) or hardware or software control logic, read only memory (ROM), and/or other types of nonvolatile memory. Additional components of computer system 14 may include one or more disk drives, one or more network ports for communicating with external devices as well as various input and output (I/O) devices, such as a keyboard, a mouse, and a video display.

In the illustrated embodiment, computer system 14 is interfaced with genetic analyzer 12 through a direct connection. The direct connection may be any cable for communicating data between computer system 14 and genetic analyzer 12. In another embodiment, computer system 14 may be interfaced with genetic analyzer 12 through network 18.

Server 16 couples to network 18. In the illustrated embodiment, server 16 includes database 17 that may be separate from or integral to server 16. Database 17 may be any type of database that stores and organizes information generated by one or more devices coupled to network 18. In one embodiment, results of a genetic test performed by genetic analyzer 12 may be stored in database 17. Once stored, the results may be viewed on computer system 14 or any other display device and/or system (not expressly shown) coupled to network 18.

In one embodiment, genetic analyzer 12 may perform a genetic test, such as a cystic fibrosis (CF) assay. The CF assay may test for the twenty-five (25) core mutations in a Pan-Ethnic panel as defined by the American College of Obstetrics and Gynecology (ACOG) and the American College of Medical Genetics (ACMG), as shown in Table 1.

TABLE 1
Primary ACOG Mutation Panel

| | | | | |
|---|---|---|---|---|
| ΔF508 | G542X | N1303K | 3659dC | 1078dT |
| ΔI507 | G551D | R117H | G85E | 1898 + 1G > A |
| W1282X | R560T | R347P | 2789 + | 621 + 1G > T |
| 1717 − | R553X | R334W | 5G > A | 3120 + 1G > A |
| 1G > A | 3849 + | I148T | 2184dA | 711 + 1G > T |
| R1162X | 10kBC > T | | A455E | |

Although a specific CF assay is described in detail below, other CF assays and other genetic tests may be performed using genetic analyzer 12 and processed by computer system 14 using a similar technique.

To perform the CF assay, patient samples of whole blood or buccal cells may be purified and placed in a multiplex PCR reaction with primers encompassing the twenty-five (25) core mutations. An aliquot of the multiplex, biotin-modified PCR reaction for each patient sample may be transferred to one of the wells included on a microtiter plate.

Color coded microspheres, also known as beads, that have specific strands of DNA attached, also known as oligonucleotides or oligos, may be added to each of the DNA samples contained in the wells. In one embodiment, approximately fifty-two different beads may be used to detect the twenty-five core (25) mutations in the primary ACOG panel. Forty-eight (48) beads may be either normal or mutant beads associated with the tested alleles. The remaining four (4) beads may be control beads used to provide a reference signal intensity in each of the patient samples that are used to calculate the results of the performed test. In other embodiments, the number of beads, either assay beads or control beads, used for any given genetic test may depend on the test being performed.

Unlike the beads used to detect the mutations, the oligo sequences on the control beads may lack homology to any DNA in rat, mouse and/or human. In one embodiment, the control beads may include a capture probe bonded to the microsphere. For example, the capture probe may be different levels of biotin covalently bonded to the microspheres. The four control beads may be identified as k1, k2, k3 and k4. The k1 bead may have no biotin bonded on the microsphere, the k2 bead may have x amount of biotin, the k3 bead may have y amount of biotin and the k4 bead may have z amount of biotin, where x<y<z. In other embodiments, the control beads may be formed by pre-binding a fluorophore directly to the bead, where each bead produces a different fluorescent signal based on the fluorophore bonded to the bead. In other embodiments, any type of coupling chemistry that creates beads having different fluorescent signals may be used.

In operation, the control beads may be used to calculate test results using fluorescence signal thresholds determined by the relative intensities of the four control beads. Each of the four (4) control beads may provide a fluorescent control signal that may be used to perform quality control tests on the equipment (e.g., genetic analyzer 12) and CF assays, to provide a minimum signal intensity for the assays and to provide a background corrected signal intensity by subtracting background signal intensity. Although the fluorescent signals have been described as being produced by four control beads including biotin covalently bonded to microspheres, any number of control beads generated by a coupling chemistry that allows for a change in signal intensity between the beads may be used.

Genetic analyzer 12 may perform the CF assay by identifying the beads associated with the tested alleles and the control beads and measuring a signal intensity associated with the identified bead. For most mutations, two beads, e.g., a normal bead and a mutant bead, may be used to respectively detect normal DNA and mutant DNA on a tested allele. The normal bead may include an oligo that searches for normal DNA and the mutant bead may include an oligo that searches for mutant DNA.

In one embodiment, genetic analyzer 12 may include two different lasers that detect the normal, mutant and control beads. The first laser may determine the bead identity, e.g., whether the bead is a normal bead, a mutant bead or a control bead, based on the fluorescence generated when the bead passes through the first laser. The second laser may detect a hybridization event associated with the identified bead. The hybridization event may occur when the second laser detects a fluorescent signal for the identified bead, which indicates that a streptavidin, also generally referred to as an exogenous target, associated with a strand of the patient DNA is attached to the oligo on the identified bead. If the laser detects a signal from a normal bead, genetic analyzer 12 categorizes the attached DNA as normal and associates the signal intensity with normal DNA for the specific allele. If the laser detects a signal from a mutant bead, genetic analyzer 12 categorizes the attached DNA as mutant and associates the signal intensity with mutant DNA for the specific allele. If the laser detects a signal from a control bead, genetic analyzer 12 categorizes the bead as a control signal and records the signal intensity for the identified control bead.

In operation, genetic analyzer 12 collects raw data (e.g., signal intensities associated with each of the normal and mutant beads) and categorizes the beads for each of the mutations in the primary panel. Genetic analyzer 12 further measures the signal intensities for the control beads and compiles the raw data and measured control bead signal intensities in a results file. In one embodiment, the results file may be a flat CSV file. In other embodiments, the results file may be any appropriate digital file that may be read and manipulated by computer system 14.

The raw data contained in the results file may include data for calculating an allele ratio associated with each of the patient samples in the wells of the microtiter plate. An allele ratio may be defined as the ratio of mutant DNA to the total DNA associated with a specific allele. The allele ratio may be calculated with the following formula:
allele ratio=x/(x+y)
where x represents the background corrected signal intensity associated with the genetic mutation on a specific allele and y represents the background corrected signal intensity associated with normal DNA on the specific allele. The allele ratio may be used to provide an allele call for each of the tested alleles.

For example, an allele call may be defined as normal (also referred to as “wild type”), or heterozygous or homozygous mutant. In one embodiment, a range of allele ratios may be associated with normal allele calls and heterozygous or homozygous mutant allele calls. For example, in a CF assay testing for mutations on one or more alleles, a normal allele call may be represented by an allele ratio of between approximately zero (0) and approximately 0.3 for one or more of the tested alleles. A heterozygous allele call may be represented by an allele ratio of between approximately 0.3 and approximately 0.7 and a homozygous allele call may be represented by an allele ratio of between approximately 0.7 and approximately one (1). For mutations on other alleles, the ranges of allele ratios for each allele call may vary. In one embodiment, the ranges of allele ratios for each allele call may be configurable by a user of system 10 and the user may assign the appropriate ranges before testing begins.

Computer system 14 may receive the results file from genetic analyzer 12 and process the raw data included in the results file. For example, computer system 14 may determine a minimum signal intensity associated with the control beads in each of the wells. The minimum signal intensity may be used to validate the control beads. If the minimum signal intensity is less than a minimum signal threshold, computer system 14 may determine that an error (e.g., the control beads were not added to the patient samples or genetic analyzer 12 malfunctioned) occurred during performance of the genetic test and the tested alleles may be marked as run failures. If the minimum signal intensity is greater than the minimum signal threshold, computer system 14 may calculate the results of the CF assay for each of the patient samples by applying the corresponding control signals to the raw data collected from the appropriate well. In one embodiment, computer system 14 may communicate the raw data and calculated results of the CF assay for each tested patient sample to database 17. Computer system 14 may then display the calculated results and the raw data in a graphical user interface (GUI).

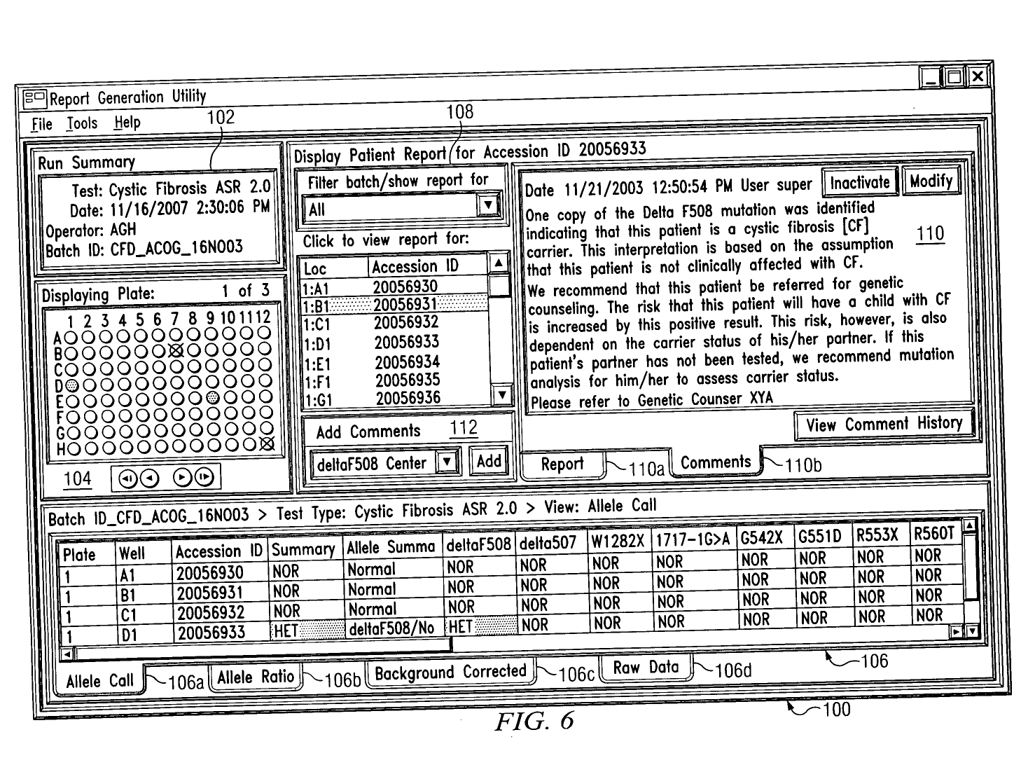

As described in more detail below in reference to FIGS. 5 through 7, the GUI may include representations of the calculated results and the raw data in multiple formats. For example, the information from the results file may be displayed in a summary report containing the calculated results and raw data for each of the patient samples tested. The GUI may also include a plate view containing a color-coded representation of the calculated results for each of the wells on the plate. The GUI may further include a patient report associated with each one of the patient samples. In one embodiment, the patient report may include a comment describing the results of the test. For example, if no CF mutations were detected, the comment may state that the patient is not a carrier of any of the core CF mutations. If one of the core mutations was identified, the comment may include an explanation of the mutation and any recommendations for the patient regarding further tests and/or genetic counseling.

In one embodiment, the GUI may further include a virtual line blot for displaying the calculated results. The virtual line blot may provide a visual representation of the calculated results that allows a user at computer system 14 to quickly interpret the results of the genetic test. For example, the virtual line blot may include a pattern recognition feature for each tested allele and the user may determine if a patient sample is normal or contains a mutation by viewing the corresponding pattern recognition feature.

Genetic analyzer 12 may further be used to perform a reflex test. The reflex test generally indicates the severity of the detected mutation on a reflex allele (e.g., a deltaF508 mutation on the F508 allele and a R117H mutation on the R117 allele). Genetic analyzer 12 may perform the reflex test using a technique similar to that described above in reference to the performance of the CF assay. Again, the raw data including the signal intensities associated with the reflex beads and the measured signal intensities for the control beads may be placed in an output file and sent to computer system 14. Computer system 14 may calculate the results of the reflex test using a technique similar to the one described above in reference to processing the CF assay raw data. The calculated results may be integrated with the results from the CF assay for the specific patient and displayed in the GUI.

In one embodiment, the reflex test may be performed separate from the genetic test only if a mutation is detected on one of the reflex alleles. The reflex test may be performed by adding control beads and reflex beads associated with the levels of severity for the specific CF mutation to each of the patient samples identified as having the specific CF mutation. For example, the test for the presence of poly T (e.g., 5T, 7T and 9T) in the presence of the R117H mutation may be performed by adding a total of seven beads to the patient samples: the four control beads used in the CF assay and three reflex beads used to test for the presence of 5T, 7T and 9T.

The results of the reflex test may be calculated as described above and computer system 14 may compile the results in for communication to database 17. The reflex results may be associated with the results for the CF assay performed on the corresponding patient sample in database 17. Computer system 14 may further integrate the reflex results with the CF assay results and display the data simultaneously in the GUI. By performing the reflex test only if a mutation is detected on a specific allele, a false positive result indicating the presence of the severity mutation without the presence of the corresponding CF mutation may be obviated.

In another embodiment, the reflex test may be performed simultaneously with the CF assay. The reflex beads may be included with the CF beads and the control beads and added to the patient samples. Genetic analyzer 12 may perform the CF assay and reflex test as described above. The raw data for the CF assay and the reflex test (e.g., the detected signal intensities for the normal and mutant CF beads and the detected signal intensities for the reflex beads) and the measured signal intensities for the control beads may be compiled into a single output file that is communicated to computer system 14.

In one embodiment, genetic analyzer 12 may parse the results of the genetic and reflex tests into separate files. In another embodiment, computer system 14 may parse the output file into multiple files. A first file may include the raw data and measured control signal intensities from the CF assay and a second file may include the raw data and measure control signal intensities from the reflex test. By splitting the output file received from genetic analyzer 12 into separate files, the results of the reflex test may be electronically masked. For example, if no CF mutations are detected on a reflex allele, the reflex results may be ignored to avoid reporting a false positive result. If a mutation is detected on a reflex allele for one of the patient samples, computer system 14 may calculate the results of the reflex test for the identified patient sample and integrate the reflex results with the CF results for the identified patient sample. The combined results may then be displayed in a GUI.

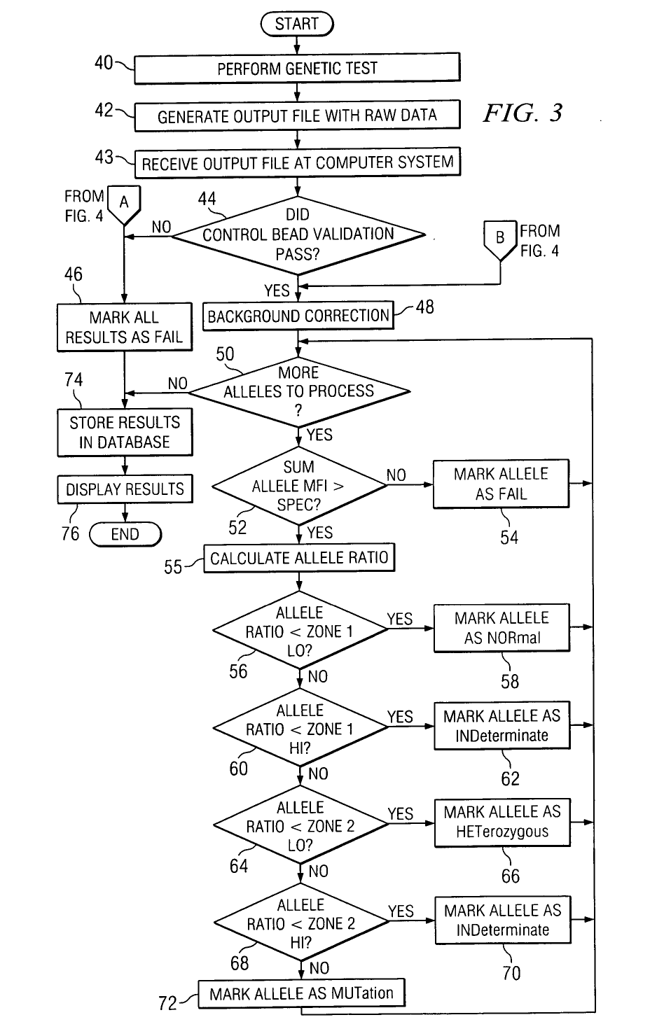

FIG. 2 illustrates a block diagram of computer system 14 used to process and display data received from genetic analyzer 12. In the illustrated embodiment, computer system 14 may include respective software components and hardware components, such as processor 20, memory 22, and hard disk drive (HDD) 24, and those components may work together via bus 34 to provide the desired functionality. The various hardware and software components may also be referred to as processing resources. Data processing service (DPS) module 26, report generation utility (RGU) module 28, data browser (DB) module 30 and administration utility (AU) module 32, which reside in memory, such as HDD 24, may be executable by processor 20 through bus 34.

Processor 20 may be a microprocessor, a microcontroller, a digital signal processor (DSP) or any other digital or analog circuitry configured to execute processing instructions stored in HDD 24. Memory 22 may be random access memory (RAM), electrically erasable programmable read-only memory (EEPROM), a PCMCIA card, flash memory, or any suitable selection and/or array of volatile or non-volatile memory.

Computer system 14 may further include display 36 for presenting a GUI and input devices such as a mouse and a keyboard (not expressly shown). Display 36 may present the GUI (described in more detail with respect to FIGS. 5-7), which allows a user to view the calculated results and raw data for a genetic test and/or a reflex test. Display device 36 may be a liquid crystal device, cathode ray tube, or other display device suitable for creating graphic images and alphanumeric characters recognizable to a user.

In operation, processing instructions are located in memory 22. These processing instructions may be stored in HDD 24 and loaded into memory 22 via bus 34. Processor 20 may accesses memory 22 to retrieve the processing instructions and perform various functions included in the processing instructions. In one embodiment, the processing instructions may include DPS module 26, RGU module 28, DB module 30 and AU module 32. DPS module 26 may provide the ability to process raw data received from genetic analyzer 12 and calculate results for the genetic and/or reflex tests performed. RGU module 28 may provide the primary user interface to the raw data and the calculated results in formats including, but not limited to, plate views, summary reports, patient reports, batch reports and virtual line blots. DB module 30 may provide a drag-and-drop interface for access to any results stored in database 17. AU module 32 may allow a user of computer system 14 to configure information for use by DPS module 26.

During testing, genetic analyzer 12 may create an output file containing the raw data for the genetic test and/or the reflex test and the measured intensities for the control signals associated with each of the patient samples in each of the wells on the plate. In one embodiment, the genetic test and the reflex test may be performed simultaneously and the output file may include the raw data associated with each test. The output file may be communicated to computer system 14 for processing where the output file may be parsed into individual files including the results of the genetic and reflex test. In another embodiment, the output file may be parsed by genetic analyzer 12 into a genetic file and a reflex file and communicated to computer system 14. In other embodiments, the genetic test and the reflex test may be performed separately and the results of each test may be included in different output files such that the results of each test may be communicated to computer system 14 in the respective output files after the test is complete. The output file may be stored in either or both of memory 22 and HDD 24.

Upon receiving the output file, processor 20 may execute DPS module 26 in order to begin processing the raw data and to calculate the results of the genetic test and/or the reflex test for the multiple different patient samples. When a single combined bead array is used to perform both the genetic test and the reflex test, the output file may be parsed to meet the configuration requirements of the software. If the genetic test and the reflex test were performed simultaneously and the received output file contains the raw data associated with both tests, DPS module 26 or genetic analyzer 12 may parse the output file into separate files: a single file containing the raw data from the genetic test and a single file containing the raw data from the reflex test. If the genetic test and the reflex test were performed separately, DPS module 26 processes the output file associated with the corresponding test when it is received from genetic analyzer 12.

DPS module 26 may validate the control beads by calculating a signal-to-noise ratio based on the relative intensities of the control beads. If the calculated signal-to-noise ratio is greater than a threshold value, DPS module 26 may validate the control beads and calculate the results of the genetic test and/or the reflex test by applying the corresponding signal intensities associated with the control beads to the raw data collected for each patient sample. In one embodiment, DPS module 26 may calculate results of the genetic test including, but not limited to, background corrected median fluorescence intensity (MFI) values for each allele, where the MFI values may be calculated based on approximately fifty (50) to approximately one-hundred (100) beads per allele, allele ratios for each allele and allele calls based on the allele ratios.

DPS module 26 further may communicate the calculated results over network 18 for storage in database 17. In one embodiment, the calculated results may include the results for the genetic test and the reflex test if the tests are performed simultaneously. In another embodiment, DPS module 26 may generate separate results for the genetic test and the reflex test if the tests are performed separately. By storing the results in database 17, various parameters associated with the performance of the genetic test may be tracked.

Although DPS module 26 may calculate the genetic test results and the reflex test results, DPS module 26 may use different logic to calculate the results of the reflex test. For example, for most mutations in the Pan-Ethnic panel, one bead may be used to detect normal DNA and another bead may be used to detect mutant DNA. In a reflex test (e.g., the reflex test associated with the R117H mutation), a single reflex bead may be used to detect the presence of a severity on the associated allele rather than using two reflex beads to detect the presence and absence of the severity on the allele. If a signal intensity is detected for any one of the three reflex beads, DPS module 28 determines that at least one of the severities is present. Even though the logic used to calculate the results of a genetic test and a reflex test may be different, DPS module 26 may be configurable to allow the results of each test to be calculated. Additionally, DPS module 26 may be configured to calculate the results of other types of genetic tests.

In one embodiment, the genetic test and/or the reflex test for a patient sample may be performed multiple times. Between the different runs, a user of computer system 14 may reconfigure one or more user defined parameters associated with the test. For example, a first run of the test on a patient sample may be performed using one set of user defined parameters and a second run of the test on the same patient sample may be performed using another set of user defined parameters. In one embodiment, the results of the two tests may be different since the user defined parameters were changed between runs. DPS module 26 stores the results of the both tests in database 17 such that the data associated with the first test is not overwritten by the data associated with the second test even though the same test was performed on the patient sample multiple times. DPS module 26, therefore, may allow the testing history associated with a patient sample to be tracked because the results of any test performed on the patient sample are maintained in database 17.

After processing by DPS module 26, the calculated results and raw data may be included in a GUI by executing RGU module 28. The GUI may display information associated with the calculated results and raw data in formats including, but not limited to, a header summary, a color-coded plate view, a summary report, a batch report, a patient report and/or a virtual line blot. RGU module 28 may allow access to the data by accession number (e.g., patient number), batch name and/or date that the test was performed. RGU module 28 additionally may provide sorting and printing capabilities such that the displayed data may be organized and printed in numerous different ways. For example, RGU module 28 may allow a user to filter the results of the genetic test based on an allele call and view the patient reports including the selected allele call. Additionally, the filter may be used to generate a virtual line blot including only the filtered results. By filtering the results, RGU module 28 may provide only the results for patient samples having the selected allele call for the user to view in the patient reports and virtual line blots displayed in the GUI. Information associated with results having a different allele call, however, may be present for viewing by the user in, for example, the header summary, the color-coded plate view, and the summary report.

If a reflex test is performed, RGU module 28 may read the results file to obtain the data associated with the reflex test. RGU module 28 may integrate the results of the reflex test into the appropriate section of the GUI by using, for example, the accession number associated with the genetic test and the reflex test.

In one embodiment, the genetic test and/or the reflex test for a patient sample associated with a specific accession number may be performed multiple times. RGU module 28 may provide the user access to the results of both tests for each time that they are performed. If the user selects to view the results of the genetic test associated with the second time that the genetic test was performed, RGU module 28 automatically links the results of the genetic test with the results of the final reflex test that was performed. For example, if the reflex test was performed three times, RGU module 28 links the results of the genetic test with the results of the reflex test associated with the third time that the reflex test was performed. Additionally, if the user selects to view one of the results of the reflex test that was performed multiple times for a specific patient sample, RGU module 28 automatically links the reflex test results with the results of the final genetic test that was performed.

When executed by processor 20, DB module 30 may provide a drag-and-drop interface for access into database 17, which includes test results generated by DPS module 26. Access to database 17 over network 18 provided by DB module 30 may allow a laboratory director or manager to track lab performance, technician performance, user performance, shift performance, the number and type of mutations detected over a specific time period and/or the trending of signal intensity in particular alleles.

AU module 32 may be executed before DPS module 16 to activate tests and configure values for use during the tests including, but not limited to, allele cutoff ratios, equivocal zones, allele names, ranges of allele ratios, scale factors, a high signal threshold, and a low signal threshold. For example, AU module 32 may allow a user of computers system 14 to configure laboratory specific information, such as Clinical Laboratory Improvement Amendments (CLIA) number, lab logo, CPT codes, and disclaimers, allow the preparation of canned comments to add into the patient reports generated by RGU module 28, establish user access and passwords for users of computer system 14, and define allele cutoff ratios and equivocal zones based on user input parameters. In one embodiment, the user passwords may be associated with the level of access that a user has to any comments included in a patient report. For example, the access levels may include viewing, adding, modifying and/or inactivating comments. In other embodiments, there may be any number of password levels that provide any suitable user controlled access to the comments contained in the patient report.

In some embodiments, the processing instructions for DPS module 26, RGU module 28, DB module 30 and AU module 32 may be encoded in computer-usable media. Such computer-usable media may include, without limitation, storage media such as floppy disks, hard disks, CD-ROMs, DVDs, read-only memory, and random access memory; as well as communications media such as wires, optical fibers, microwaves, radio waves, and other electromagnetic or optical carriers.

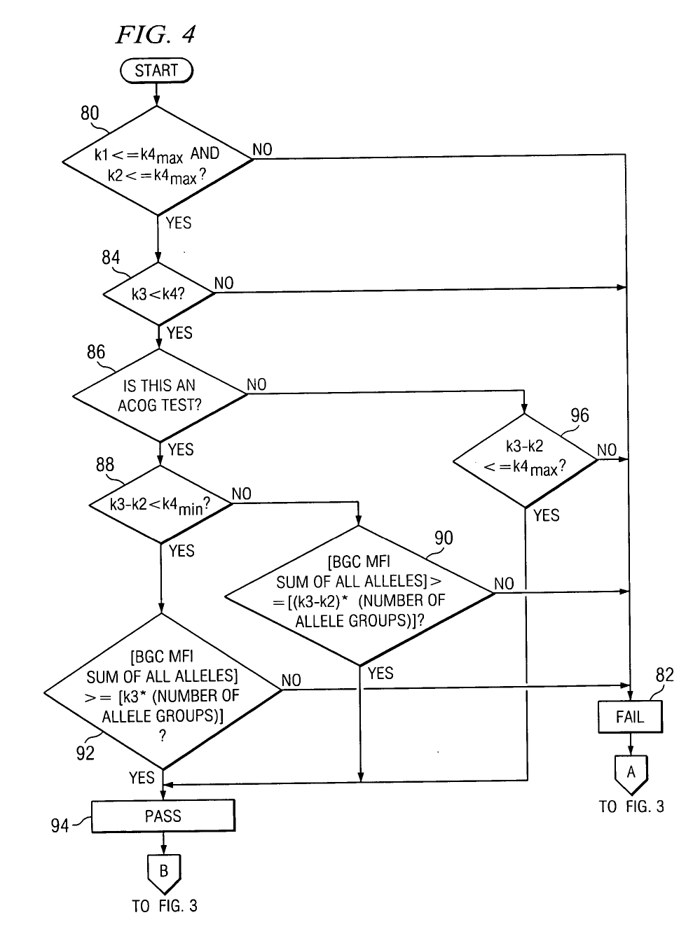

FIG. 3 illustrates a flow chart of a method for processing results of a genetic test. Generally, a genetic test, such as a CF assay, may be performed on multiple patient samples included in wells of a microtiter plate using genetic analyzer 12. Raw data collected by genetic analyzer 12 may be included in an output file communicated to computer system 14. Computer system 14 may calculate results of a genetic test for each patient sample by applying the ratio of signal intensities determined from the fluorescent control signals to the raw data associated with each well of a plate. Computer system 14 may then display the results using multiple formats in a graphical user interface.

At step 40, genetic analyzer 12 may be used to perform a genetic test. In one embodiment, the genetic test may be a CF assay that tests for twenty-five core mutations in a Pan-Ethnic panel determined by ACOG and ACMG. In other embodiments, the genetic test may be any test using a single-well multiplex analysis. The CF assay may be performed by placing DNA samples from multiple patients in wells included on a microtiter plate. The CF assay may be performed as described above in reference to FIG. 1.

At step 42, genetic analyzer 12 may generate an output file including raw data associated with the CF assay performed. In one embodiment, the output file may be a digital file that allows the analysis of the raw data to be performed electronically. The raw data may include signal intensities associated with the CF beads that detect normal and mutant DNA and signal intensities associated with the control beads. The output file may be communicated to computer system 14 for processing by DPS module 26 at step 43.

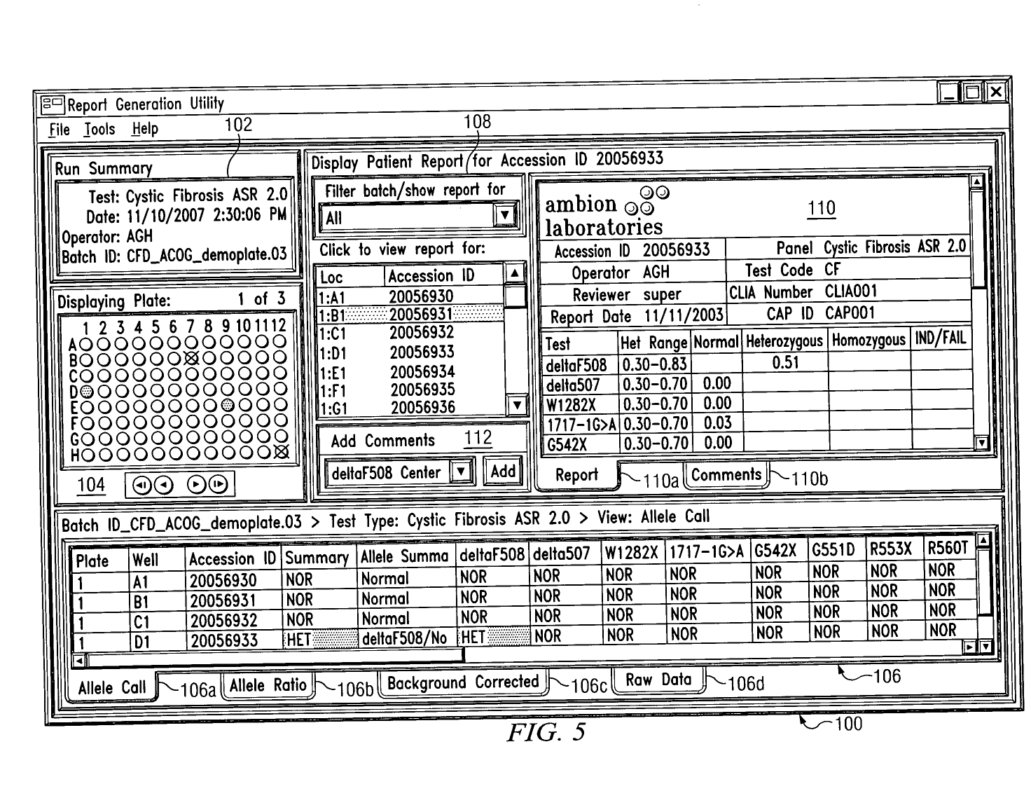

DPS module 26 in computer system 14 may begin processing the raw data by determining whether the signal intensities of the control beads passed a validation test at step 44. One example embodiment of a validation test for the control beads is described in detail below with respect to FIG. 4. If the control beads do not pass the validation test, DPS module 26 marks all of the test results as failures at step 46.

If the control beads pass the validation test, DPS module 26 adjusts the raw data based on the background intensity for each of the wells to generate background corrected signals at step 48. The background intensity in each well may affect the relative signal intensities measured by genetic analyzer 12. In order to provide the correct results, the background intensity may be removed from any calculations using the raw data collected by genetic analyzer 12. In one embodiment, the background intensity may be removed by subtracting the measured intensity for the k2 control bead from the total detected intensity for each normal and mutant bead associated with the tested alleles. The background corrected intensities for each of the normal and mutant beads may then be used by DPS module 26 to calculate the results of the CF assay.

At step 50, DPS module 26 determines if the results for all alleles have been processed. If DPS module 26 has processed all tested alleles, DPS module 26 determines if the sum of the MFI values for the normal beads and the mutant beads associated with one of the alleles is greater than a minimum signal threshold at step 52. The sum of the MFI values for a selected allele may be calculated by adding the detected signal intensities for the mutant and normal beads. Since the control beads are spiked into each well, the control beads may provide well-to-well specific correction. The standard deviation of the control bead signals across multiple wells may provide the minimum threshold noise level in the system. In one embodiment, the minimum threshold may be calculated for a specific allele by subtracting the measured signal intensity for the k2 control bead from the measured signal intensity for the k3 control bead for that specific allele.

If the sum of the MFI values for the selected allele is less than the minimum signal threshold, DPS module 26 marks the allele as failing at step 54. If the sum of the MFI values for the selected allele is greater than the minimum signal threshold, DPS module 26 calculates an allele ratio for the selected allele at step 55.

At step 56, DPS module 26 compares the calculated allele ratio to a lower zone boundary of a first equivocal zone defined by a user of system 10. In one embodiment, an equivocal zone may define a range of allele ratios that indicate an indeterminate result for the CF assay on the tested allele. The indeterminate result may represent an allele ratio that is within a predetermined amount cutoff ratios associated with the different allele calls. The width of the equivocal zone and allele ratios included in the equivocal zone may be configurable a user of system 10 for each tested allele.

The first equivocal zone may be defined as the region surrounding the cutoff ratio for a normal ratio range. For example, the cutoff ratio for a specific allele may be approximately 0.3. The first equivocal zone may be defined by a lower zone boundary and an upper zone boundary that are approximately equidistant from the cutoff ratio for the normal ratio range. If the calculated allele ratio is less than the lower zone boundary for the first equivocal zone, DPS module 26 marks the selected allele as normal and records the normal test result for the selected allele at step 58. DPS module 26 then determines if any alleles remain for processing at step 50.

If the calculated allele ratio is greater than the lower zone boundary of the first equivocal zone, DPS module 26 determines if the allele ratio is located in the first equivocal zone at step 60. If the allele ratio is less than the upper zone boundary for the first equivocal zone, DPS module 26 marks the selected allele as indeterminate and records the indeterminate result for the selected allele in the result file at step 62. DPS module 26 then determines if any alleles remain for processing at step 50.

If the calculated allele ratio is greater than the upper zone boundary for the first equivocal zone, DPS module 26 determines if the calculated allele ratio is less than a lower zone boundary associated with a second equivocal zone at step 64. In one embodiment, the second equivocal zone may be defined as the region surrounding the cutoff ratio for a heterozygous ratio range. For example, the cutoff ratio for a heterozygous ratio range may be approximately 0.7. The second equivocal zone may be defined by a lower zone boundary and an upper zone boundary that are approximately equidistant from the cutoff ratio for the heterozygous ratio range. If the calculated allele ratio is less than the lower zone boundary for the second equivocal zone, DPS module 26 marks the selected allele as heterozygous and records the heterozygous result for the selected allele in the results file at step 66. DPS module 26 then determines if any alleles remain for processing at step 50.

If the calculated allele ratio is greater than the lower zone boundary of the second equivocal zone, DPS module 26 determines if the allele ratio is located in the second equivocal zone at step 68. If the allele ratio is less than the upper zone boundary for the second equivocal zone, DPS module 26 marks the allele as indeterminate and records the indeterminate result for the selected allele in the results file at step 70. DPS module 26 then determines if any alleles remain for processing at step 50. If the calculated allele ratio is greater than the upper zone boundary for the second equivocal zone, DPS module 26 marks the allele as MUT for homozygous mutant and records the result for the selected allele in the results file at step 72. DPS module 26 then determines if any alleles remain for processing at step 50.

The above steps 50-72 may be repeated by DPS module 26 for each allele tested in the CF assay. When DPS module 26 has processed all alleles tested or DPS module 26 has marked all results as fail because the control beads did not pass validation, DPS module 26 communicates the calculated results and raw data for the tests performed by genetic analyzer 12 to database 17 over network 18 at step 74. RGU module 28 queries the database created by DPS module 26 and creates a GUI for displaying the results at step 76. As described below in reference to FIGS. 5 through 7, the results of the CF assay may be displayed in multiple formats within one GUI and/or multiple formats within multiple GUIs.

Although not described in detail with respect to FIG. 3, a reflex test may be performed similar to the CF assay as described by steps 40-76 using separate reflex beads. In one embodiment, the reflex test may be performed separate from the CF assay. A separate output file may be generated by genetic analyzer 12 that contains the raw data for the reflex test and the measured signal intensities for the control beads. The output file may be processed by DPS module 26 and the results of the reflex test may be calculated by determining ratios associated with the signal intensities for the reflex beads.

In another embodiment, the reflex test may be performed simultaneously with the CF assay. Genetic analyzer 12 may generate separate output files for the CF assay and reflex test and communicate the separate files to computer system 14. The individual output files may include raw data for the respective test and the measured signal intensities for the control beads. In a further embodiment, the output file created by genetic analyzer 12 may include raw data for the CF assay, raw data for the reflex test and the measured signal intensities for the control beads. DPS module 26 may receive the output file and parse the file into smaller files. For example, DPS module 26 may create a CF file that includes the raw data from the CF assay and a reflex file that includes the raw data from the reflex test, where the measured signal intensities for the control beads are copied into the both of the CF and reflex files. Each file may be appropriately processed as described above in steps 44-72.

FIG. 4 illustrates a flow chart for one example embodiment of a validation test for control beads included in each of the patient samples tested in a CF assay. Generally, genetic analyzer 12 measures the signal intensities for each control bead during a genetic test and/or a reflex test. The measured intensities are communicated to DPS module 26 in computer system 14. DPS module 26 compares the measured intensities of the control signals with each other and/or various predetermined thresholds to determine if the genetic test and/or reflex test was performed successfully. If the control beads pass the validation test, DPS module 26 calculates the results of the genetic test and/or the reflex test based on raw data associated with each test. Although a specific number of comparisons are illustrated with respect to FIG. 4, any one or any combination of the comparisons may be used to validate the control beads.

At step 80, the measured signal intensities for the k1 and k2 control beads are compared to the high signal threshold value (e.g., k4ₘₐₓ). The high signal threshold may be configurable by a user of computer system 14 and may have a range of approximately four-hundred (400) to approximately eight-hundred (800). If the measured signal intensity for the k1 or k2 control bead is greater than the high signal threshold value, DPS module 26 determines that the validation failed at step 82. DPS module 26 then marks the results for all alleles as failures at step 46 as illustrated in FIG. 3. If the measured signal intensity for the k1 or k2 control beads is less than or approximately equal to the high signal threshold value, DPS module 26 determines if the measured intensity of the k4 control bead is greater than the measured signal intensity of the k3 control bead at step 84.

If the measured signal intensity of the k3 control bead is greater than or equal to the measured signal intensity of the k4 control bead, DPS module 26 determines that the validation failed at step 82. DPS module 26 then marks the results for all alleles as failures at step 46 as illustrated in FIG. 3. If the measured signal intensity of the k3 control bead is less than the measured signal intensity of the k4 control bead, DPS module 26 determines if the genetic analyzer 12 performed a CF assay using a primary panel of mutations, such as the twenty-five mutations determined by the ACOG and ACMG, at step 86.

If genetic analyzer 12 performed a CF assay, DPS module 26 may continue the validation process by determining if the difference between the measured signal intensity for the k3 control bead and the measured signal intensity for the k2 control bead is less than the low signal threshold (e.g., k4 min) at step 88. The low signal threshold may be configurable by a user of computer system 14 and may have a range of approximately fifty (50) to approximately two-hundred (200). If the difference between the measured signal intensities of the k3 and k2 control beads is greater than the low signal threshold, DPS module 26 determines if the sum of the MFI values for all alleles tested is greater than or approximately equal to the difference of the measured signal intensities of the k3 and k2 control beads multiplied by the number of allele groups tested at step 90. In one embodiment, the difference between the measured signal intensities of the k3 and k2 control beads may represent the signal-to-noise ratio, also referred to as the low signal threshold, associated with the test performed by genetic analyzer 12.

If the sum of the MFI values for all alleles tested is less than the signal-to-noise ratio multiplied by the number of allele groups tested, DPS module 26 determines that the validation failed at step 82. DPS module 26 then marks the results for all alleles as failures at step 46 as illustrated in FIG. 3. If the sum of the MFI values for all alleles tested is greater than the signal-to-noise ratio multiplied by the number of allele groups tested, DPS module 26 determines that the validation passed at step 94. DPS module 26 then calculates the background corrected data at step 48 as illustrated in FIG. 3.

If the difference between the measured signal intensities of the k3 and k2 control beads is less than the low signal threshold, DPS module 26 determines if the sum of the MFI values for all alleles being tested is greater than or approximately equal to the measured signal intensity of the k3 control bead multiplied by the number of allele groups tested at step 92. If the sum of the MFI values for all alleles tested is less than the measured signal intensity of the k3 control bead multiplied by the number of allele groups tested, DPS module 26 determines that the validation failed at step 82. DPS module 26 then marks the results for all alleles as failures at step 46 as illustrated in FIG. 3. If the sum of the MFI values for all alleles tested is greater than the measured signal intensity of the k3 control bead multiplied by the number of allele groups tested, DPS module 26 determines that the validation passed at step 94. DPS module 26 then calculates the background corrected data at step 48 as illustrated in FIG. 3.

If DPS module 26 determines that the output file received from genetic analyzer 12 includes raw data for a reflex test, DPS module 26 determines if the difference between the measured signal intensities for the k3 and k2 control beads is less than or equal to the high signal threshold at step 96. If the difference is greater than the high signal threshold, DPS module 26 determines that the validation failed at step 82. DPS module 26 then marks the results for all alleles as failures at step 46 as illustrated in FIG. 3. If the difference is less than or approximately equal to the high signal threshold, DPS module 26 determines that the validation passed at step 94. DPS module 26 then calculates the background corrected data at step 48 as illustrated in FIG. 3.

FIG. 5 illustrates an example embodiment of graphical user interface (GUI) 100 displaying results of a CF assay. GUI 100 may be generated when processor 20 executes RGU module 28 after processing the raw data in an output file received from genetic analyzer 12. The information displayed in GUI 100 may additionally be contained in a results file created by DPS module 26. In the illustrated embodiment, GUI 100 includes header summary 102, plate view 104, summary report 106, batch select 108, patient report 110 and comment select 112. In other embodiments, GUI 100 may be used to display any combination of data and/or results generated by DPS module 26. GUI 100 may also display results of a reflex test performed if the results of the CF assay indicate the presence of a specific CF mutation.

Header summary 102 may include identification information associated with performance of the CF assay. For example, header summary 102 may include, but is not limited to, the name of the specific test being performed, the date and time that the test was initiated by an operator, the identity of the operator performing the test and the batch identification number assigned to the specific run. This information may allow a user viewing GUI 100 to easily identify various basic information about the test.

Plate view 104 may be a chart including a color-coded representation of each well on a plate. Plate view 104 may provide an easy way to review the results of a CF assay and/or a reflex test for the patient samples contained in each of the wells. In one embodiment, a plate may include ninety-six (96) different wells that each contain a sample of blood from different patients. The plate may include eight rows, labeled A through H, and twelve columns, numbered 1 through 12. In other embodiments, the plate may include any number of wells and may be configured to have any number of rows and columns that provide the desired number of wells. Plate view 104 may include multiple plates that may be displayed in GUI 100 simultaneously or individually. In one embodiment, multiple plates may be identified by detecting results calculated from a patient sample provided in the A1 well.

In the illustrated embodiment, plate view 104 includes the results of the CF assay for each of the wells. For example, plate view 104 indicates whether the results of the CF assay were normal, a genetic mutation was found, the results were indeterminate and/or the test failed. Normal results are shown with a non-shaded circle. For example, the results of the CF assay performed on the patient sample in well A1 were normal. In one embodiment, normal results may be indicated by the color green when viewed on display 36.

Mutant results, where the patient is identified as either heterozygous or homozygous, and indeterminate results are shown with a shaded circle. For example, the CF assay performed on the patient sample in well D1 detected mutant DNA for one of the alleles tested. In other embodiments, the shaded circle may represent an indeterminate result, which indicates that a calculated allele ratio is close to an allele ratio cutoff (e.g., the highest allele ratio associated with a specific result) for either a normal result or a heterozygous result. In one embodiment, the color yellow may indicate mutant or indeterminate results when viewed on display 36.

A run failure indicating that the genetic test was not properly performed is shown with an X over a circle. For example, the CF assay performed on the patient sample in well B7 failed and no raw data was generated. The failure could indicate any number of problems including, but not limited to, a failure to place a patient sample in the well, a failure to add the panel beads and/or the control beads to the patient sample, a failure in genetic analyzer 12 and/or a failure to validate the control beads. In one embodiment, the color red may indicate the failure when viewed on display 36 and the X may provide a way for a color blind user to differentiate between a well including a patient sample with normal results and a well including a run failure.

Summary report 106 may include one or more charts showing the results of the CF assay and/or the reflex test as processed by DPS module 26. The charts may include, but are not limited to, information such as plate number, the well number corresponding to a patient sample, the accession identification number, also referred to as the patient number, associated with the individual patient samples in the wells, a summary of the overall test results and a summary of the results for each genetic mutation and/or reflex severity tested.

Tab 106a may include information related to the allele call calculated by DPS module 26. The allele call may indicate whether the patient tested positive for a CF mutation on a specific allele. A normal result may be indicated by “NOR”, a mutant result where the patient is found to have CF may be indicated by “MUT”, a mutant result where the patient is found to be a carrier of CF (e.g., the patient has mutant DNA and normal DNA associated with at least one allele) may be indicated by “HET”, an indeterminate result may be indicated by “IND” and a run failure may be indicated by “FAIL”. In one embodiment, tab 106a may further be color-coded based on the colors used in plate view 104. For example, a heterozygous result may be detected in well D1. The text included in the allele summary column and the deltaF508 column may be highlighted using the same color (e.g., yellow) used in plate view 104 to allow for easy identification of patients who are carriers of a CF mutation or who may have CF.

Tab 106b may include information related to the allele ratio calculated by DPS module 26. As described above in reference to FIG. 1, the allele ratio may be defined as the ratio of mutant DNA to the total DNA associated with a specific allele. The allele ratio may be used to generate the allele call for each of the tested alleles.

In one embodiment, a range of allele ratios may be associated with a normal result, a heterozygous result and a homozygous result. For example, in a cystic fibrosis assay testing for a mutation on the W1282X allele, the normal ratio range may be between approximately zero (0) and approximately 0.3, where 0.3 is the allele ratio cutoff for the normal ratio range, the heterozygous ratio range may be between approximately 0.3 and approximately 0.7, where 0.7 is the allele ratio cutoff of the heterozygous ratio range, and the homozygous ratio range may be between approximately 0.7 and approximately one (1). For mutations on other alleles, the ranges of allele ratios for each allele call may vary. In one embodiment, the ranges of allele ratios for each allele call may be configurable by a user of system 10 and the user may assign the appropriate ranges before testing begins. For most alleles, a ratio of approximately 0.5 may indicate a heterozygous mutation in the tested patient but the value may be higher or lower depending on the ranges assigned by the user.

The allele ratio ranges may also be used to determine if the results are indeterminate. For example, if the calculated allele ratio is too close to the cutoff ratios for the various ranges, the results of the assay may be indeterminate. Using the W1282X allele as an example, if the allele ratio is calculated by DPS module 26 to be 0.3, the results may be indeterminate since the ratio is located on the border of the normal and heterozygous ranges. Whether the results of the CF assay are indeterminate may further depend on user defined equivocal zones. In one embodiment, a user may configure the width and/or the allele ratios included in the equivocal zones for each tested allele and/or each genetic test performed. These zones may be set when processor 20 executes AU module 32. An equivocal zone may be an area surrounding the limits of the different ranges where, if a ratio is in the area, the results may be indeterminate. Again, using the W1282X allele as an example, an equivocal zone may be defined by the areas between approximately 0.25 and approximately 0.35. If the allele ratio calculated by DPS module 26 falls between these two values, the results of the CF assay may be indeterminate.

Tab 106c may include information related to the background corrected MFI values, also referred to as normalized results. As described above in reference to FIG. 1, each of the patient samples may include one or more control beads that have different amounts of biotin covalently bonded to the microsphere. The background intensity in each well may affect the relative signal intensities measured by genetic analyzer 12. In order to provide the correct allele ratio, the background intensity may be removed from the calculation. In one embodiment, the background intensity may be removed by subtracting the intensity associated with the k2 control bead from the detected intensity of the normal and mutant beads associated with each tested allele. The background corrected signal intensities for each of the alleles included in the background corrected chart may then be used by DPS module 26 to calculate the allele ratio and allele call for the different mutations and generate a virtual line blot.

Tab 106d may include the raw data collected by genetic analyzer 12. The raw data may include the signal intensities measured for the mutant, normal and/or reflex beads associated with each of the alleles tested. Although the raw data may not be used to directly calculate the results of the CF assay, the raw data may be normalized as described above and then used to calculate the results displayed in other parts of GUI 100.

Batch select 108 may allow a user to filter the results to view in GUI 100. In the illustrated embodiment, all of the results associated with the CF assay run on a specific plate and/or plates as shown in header summary 102 may be displayed in GUI 100. In another embodiment, the user may filter the results of the genetic tests performed on the different patient samples by selecting to view the patient reports associated with a specific allele call. For example, the allele calls calculated by DPS module 26 may include normal, heterozygous, homozygous, indeterminate or run failure. By using batch select 108, the user may select to view patient reports in GUI 100 associated with patient samples where the results of the test were normal. Batch select 108 may further display the accession numbers associated with the patient reports for the filtered results. Similar to results displayed in GUI 100, batch select 108 may allow a user to print out the patient reports having a specific allele call and/or generate a virtual line blot for the patient samples with the specific allele call.

GUI 100 may also include patient report 110 associated with each of the patient samples tested. Patient report 110 may contain information including, but not limited to, the patient identification number (e.g., accession number), operator information for the person running the test on genetic analyzer 12, reviewer information for the person reviewing the results of the CF assay and/or the reflex test for the particular patient sample, the CF assay and/or reflex test performed by genetic analyzer 12, a CLIA number associated with the lab performing the CF assay and/or reflex test and the results of the CF assay and/or reflex test for each of the tested mutations. The operator information and reviewer information may be initials, a last name, an email user name or any other suitable information that identifies the person running the test using genetic analyzer 12 and the person reviewing the results of the test performed.

The patient reports available to be viewed in GUI 100 by a user may depend on the results selected in batch select 108. In the illustrated embodiment, all of the patient reports associated with the patient samples tested are selected by the user for viewing. In another embodiment, only the patient reports associated with certain allele calls (e.g., normal, heterozygous, homozygous, indeterminate and failure) may be selected for viewing. The specific patient report may be viewed in GUI 100 by selecting the accession number associated with the patient report in batch select 108.

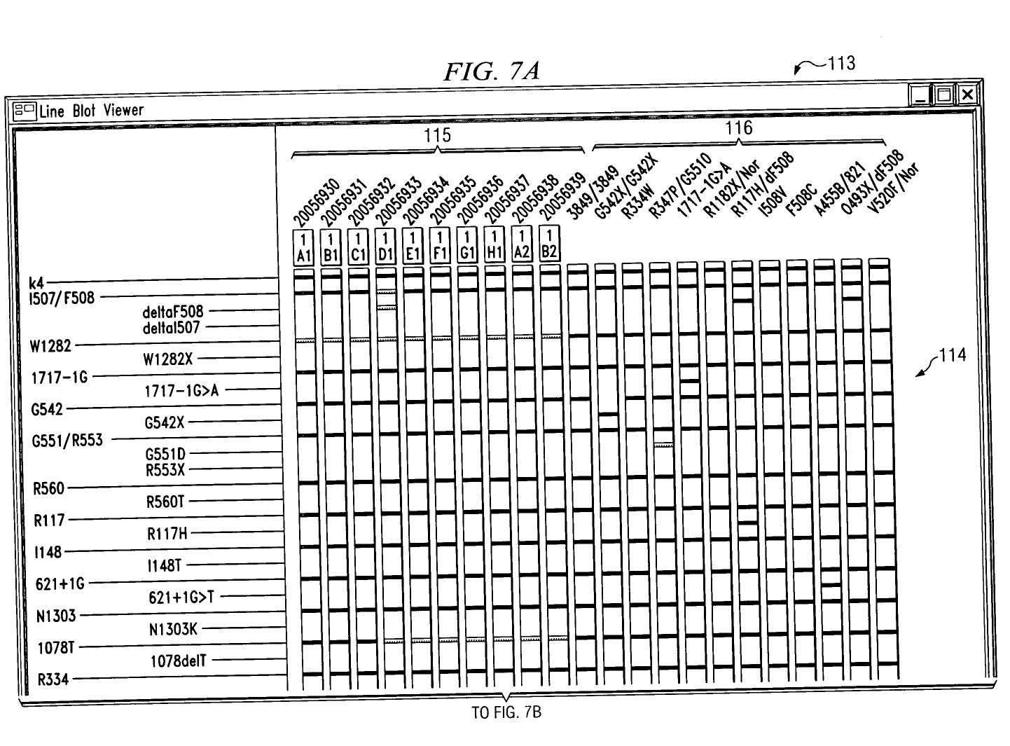

Patient report 110 may further include tabs 110a and 110b. Tab 110a may include the identification information and the results of the CF assay and/or the reflex test for the patient sample selected in batch select 108 and tab 110b may include any comments associated with the results of the CF assay for the selected patient sample. The comments may be added to patient report 110 by selecting the appropriate comments using comment select 112. As described in further detail with respect to FIG. 6, comment select 112 may allow the user to add predefined comments for a specific result or create a custom comment. In the illustrated embodiment, patient report 110, identified by accession number 20056933, indicates that the results of the CF assay for the corresponding patient sample show that the patient is a carrier of the CF mutation on the deltaF508 allele. The user may select to add a predefined comment for the deltaF508 mutation or may select to add a custom comment for the identified mutation.

For some mutations, including the deltaF508 mutation and the R117H mutation, a reflex test may be performed to determine the severity of the mutation. As described above, the results of the reflex test may be calculated by DPS module 26 and integrated into GUI 100 by RGU module 28. For example, RGU module 28 may use the accession number associated with the patient sample to combine the results of the reflex test with the results of the CF assay. The reflex test results may be displayed in one or more of plate view 104, summary report 106 and patient report 110. Additionally, batch select 108 may be used to select results where a reflex test was performed.

FIG. 6 illustrates GUI 100 displaying a comment included in tab 110b of patient report 110. As described above, the comment may be added to patient report 110 using comment select 112. In the illustrated embodiment, the comment includes the results of the CF assay (e.g., the patient is a carrier of a gene know to cause CF), recommendations for actions to be taken by the patient in the future and information indicating when the comment was added to patient report 110. In other embodiments, the comment may include any information associated with the results of a genetic test and/or reflex test performed on the corresponding patient sample. The comment may be added by any qualified person including, but not limited to, a lab director and a medical director.

As described above, a reflex test may be performed to determine the severity of, for example, a deltaF508 mutation. After the reflex test results are integrated into GUI 100, the comment associated with patient report 110 may be modified to include information related to the results of the reflex test. For example, the information may include, but is not limited to, the severity of the mutation, the time and date that the reflex test was performed and any recommendations based on the results of the reflex test. The additional information may be added using a predefined comment or a custom comment.

In some embodiments, a password may be assigned to each user of GUI 100. The password may enable the user to have different levels of access to the information contained in the comment. In one embodiment, there may be three levels of passwords. A first level password may allow a user to view the comment associated with patient report 110. A second level password may allow a user to view the comment and add a comment. For example, the user may add a predefined comment and/or a custom comment based on the results of the CF assay to patient report 110.

A third level password may allow the user to view, add, modify and inactivate the comment. For example, a comment may indicate that the run associated with the patient sample failed and/or the results of the test were indeterminate and that the sample should be tested again. If the patient sample is tested a second time and the test is performed successfully, the original comment may be inactivated and a new comment based on the results of the test may be added to patient report 110. Although the original comment may not be available for viewing in patient report 110, the comment history associated with the specific patient sample may include the inactivated comment. In one embodiment, the comment history may be stored in database 17 and associated with the appropriate accession number within the database. The comment history may include the date and time that the comment was added, modified or inactivated, the identity of the person making the change to the comment, and how the comment was modified. The comment may be modified or inactivated by clicking on the respective modify or inactivate buttons in GUI 100, by selecting the options from a pull down menu and/or by entering a command through an input device interfaced with computer system 14.

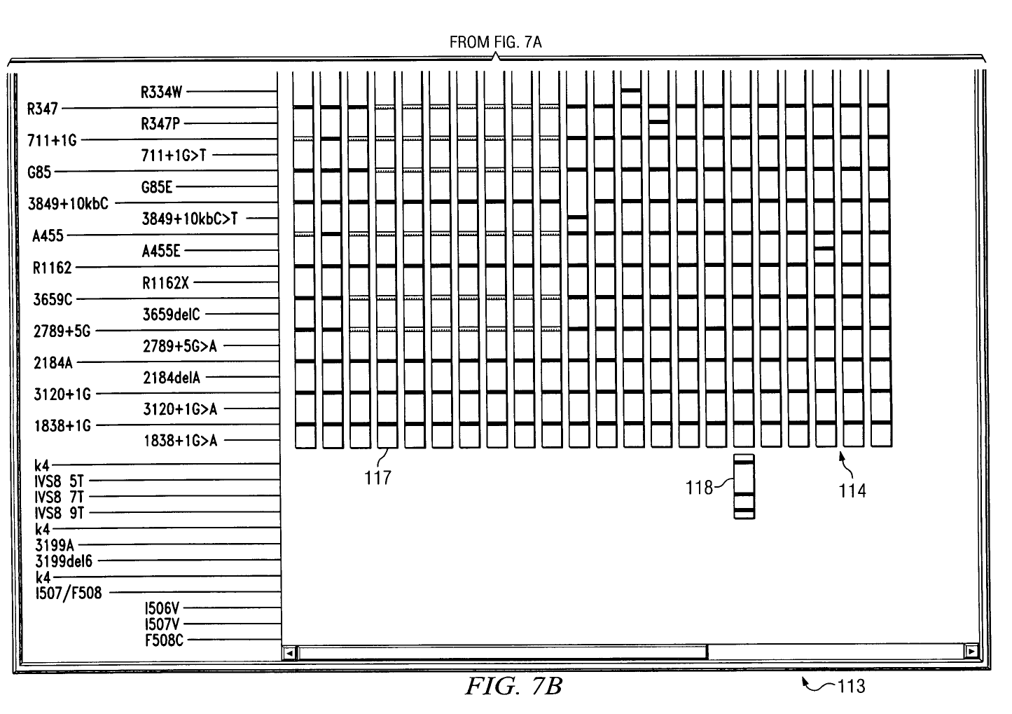

FIGS. 7A and 7B illustrate a graphical user interface including a pattern recognition tool displayed as a virtual line blot that contains the results of a CF assay. In the illustrated embodiment, GUI 113 may include virtual line blot 114. GUI 113 may be displayed separate from or simultaneous with GUI 100 as described in reference to FIGS. 5 and 6. Virtual line blot 114 provides a graphical representation of the results of the CF assay and/or reflex test so that large arrays of data may be viewed quickly by a user. As shown, the alleles tested may be listed along one side of virtual line blot 114. Two color bands may be used to represent the normal DNA and the mutant DNA associated with each allele. A pattern may be created based on the color bands and the pattern may provide a pattern recognition feature representing the results of the CF assay for each patient sample.

In the illustrated embodiment, CF results 115 represent the results of the CF assay for different patient samples. These results may correspond with the results illustrated in plate view 104 in FIGS. 5 and 6. CF patterns 116 represent the patterns formed if a mutation is detected on a specific allele. For example, if a heterozygous mutation is detected on the F508 allele, the corresponding CF pattern may include a color band representing the normal allele and a color band representing the mutant allele. A color band for the k4 control bead may also be included as a control measure.

In one embodiment, RGU module 28 may convert the calculated results for the CF assay into virtual line blot 114. To create virtual line blot 114, AU module 32 may assign a MFI scale factor to each allele. The scale factor may represent a maximum MFI value for an allele and may be configurable by a user of system 10. The scale factor may initially be assigned to a background corrected signal that represents a patient sample including only normal DNA. The signal may be represented on line blot 114 as a color band on the normal allele having an intensity of approximately one hundred percent (100%). As the signal intensity of the normal bead decreases and the signal intensity of the mutant bead increases, the color intensity for the normal and mutant bands may be adjusted to graphically represent the calculated allele ratio using a logarithmic scale based on the scale factor for the specific allele.

For example, when an individual color band is drawn for a specific allele, RGU module 28 may divide the measured background corrected allele MFI value for the patient sample by the scale factor for the allele, which generates a saturation ratio. If the saturation ratio is greater than or equal to approximately one (1), the color band is drawn with the darkest possible color. The darkest color may represent a signal intensity associated with the measured bead (e.g., normal, mutant or control bead) of approximately one-hundred percent (100%). If the saturation ratio is approximately zero (0), the color band for that allele is not visible, which may represent a signal intensity associated with the measured bead of approximately zero percent (0%). As the ratio progresses from zero (0) to one (1), the color band becomes darker based on the logarithmic scale. Therefore, if a color band for a particular allele should be darker, the displayed color intensity for the allele may be increased by decreasing the scale factor associated with the allele. Conversely, if a color band for an allele should be lighter, the displayed color intensity may be decreased by increasing the scale factor associated with the allele.

In the illustrated embodiment, patient sample 117 corresponds to well D1 as shown in plate view 104 of FIG. 5. The results of the CF assay for patient sample 117 indicate the presence of the deltaF508 mutation. As shown in patient report 110, the allele ratio was calculated to be approximately 0.51. In one embodiment, a MFI scale factor of approximately 1000 may be assigned to the F508 allele as a default value or as a value configured by a user. For an allele ratio of 0.51, the color band for the normal DNA may have an intensity of approximately fifty percent (50%) and the color band for the mutant DNA may have an intensity of approximately fifty percent (50%). As shown in patient sample 117, the color bands for the normal and mutant DNA have been adjusted to represent the appropriate intensities on each of the normal and mutant alleles. RGU module 28 calculates the color bands by logarithmically adjusting the saturation ratio associated with the normal and mutant beads between the normal and mutant color bands on virtual line blot 114. RGU module 28 may use the results calculated by DPS module 26 to display line blot 114 in GUI 113.

The same principles described with respect to a virtual line blot for a CF assay may also apply to the results of a reflex test. For example, if a R117H mutation is detected, a reflex test to determine the severity on the poly T regions may be performed. As described above, the reflex test may be performed separate from or simultaneously with the CF assay. Allele ratios for the 5T, 7T and 9T results may be calculated by DPS module 26. AU module 32 may additionally assign a MFI scale factor to each of the poly T regions, as described above in reference to the scale factor assigned for the allele groups tested in a CF assay. Color bands, similar to the bands illustrated in reflex pattern 118, may be determined by calculating a saturation ratio associated with each of the reflex beads used to identify the different poly T mutations and applying a logarithmic scale to the saturation ratio. Reflex pattern 118 may then be integrated into virtual line blot 114 by RGU module 28.

Although the present invention has been described with respect to a specific preferred embodiment thereof, various changes and modifications may be suggested to one skilled in the art and it is intended that the present invention encompass such changes and modifications fall within the scope of the appended claims.

What is claimed is:

1. A method for processing results of a complex genetic test, comprising:
receiving raw data from a genetic analyzer operable to perform a genetic test on a plurality of patient samples contained in a plurality of wells on a plate, the patient samples including at least one control bead operable to provide at least one fluorescent control signal;
calculating results of the genetic test by applying a signal intensity associated with the fluorescent control signal to the raw data; and
generating a user interface operable to display the results associated with each of the patient samples.

2. The method of claim 1, further comprising the control bead operable to define at least one of a minimum signal intensity associated with each of the wells, quality control metrics for the genetic test, and quality control metrics associated with the patient sample.

3. The method of claim 1, further comprising the control bead operable to indicate if the genetic test was performed successfully.

4. The method of claim 1, further comprising the patient samples including at least two control beads, each of the control beads operable to provide a unique signal intensity.

5. The method of claim 4, further comprising the control beads including a capture probe covalently bonded to a microsphere, the capture probe operable to hybridize with an exogenous target contained in the patient samples.

6. The method of claim 1, further comprising the genetic test operable to detect a plurality of genetic mutations.

7. The method of claim 1, wherein the genetic test comprises a cystic fibrosis assay.

8. The method of claim 1, further comprising:
receiving reflex raw data from the genetic analyzer if the results of the genetic test for at least one of the patient samples indicate a specific genetic mutation, the reflex test operable to determine a severity associated with the specific genetic mutation;
calculating results of the reflex test by applying the signal intensity associated with the fluorescent control signal to the reflex raw data; and
integrating the results of the reflex test with the results of the genetic test in the user interface for the at least one of the patient samples.

9. The method of claim 1, further comprising:
simultaneously receiving the raw data from the genetic test and reflex raw data from a reflex test from the genetic analyzer, the reflex test operable to determine a severity associated with a specific genetic mutation;
calculating results of the reflex test by applying the signal intensity associated with the fluorescent control signal to the reflex raw data if the results of the genetic test for at least one of the patient samples indicate the specific genetic mutation; and
integrating the results of the reflex test with the results of the genetic test in the user interface for the at least one of the patient samples.

10. The method of claim 9, further comprising:
receiving an output file including the raw data for the genetic test, the reflex raw data for the reflex test and the signal intensity associated with the fluorescent control signal; and
parsing the output file to generate:
a mutation file including the raw data for the genetic test and the signal intensity associated with the fluorescent control signal; and
a reflex file including the reflex raw data for the reflex test and the signal intensity associated with the fluorescent control signal.

11. The method of claim 1, further comprising:
validating the control bead; and
calculating the results of the genetic test if the control bead is validated.

12. The method of claim 1, wherein calculating the results of the genetic test comprises calculating background corrected data from the raw data by using the signal intensity associated with the fluorescent control signal.

13. The method of claim 12, further comprising the user interface including a virtual line blot generated based on the background corrected data, the virtual line blot operable to provide a visual representation of the results of the genetic test.

14. The method of claim 12, further comprising calculating at least one of an allele ratio or an allele call based on the background corrected data.

15. The method of claim 14, further comprising the user interface including:
a summary chart containing at least one of the raw data for the genetic test, the background corrected data, the allele ratio and the allele call associated with each of the patient samples; and
a plate chart including a color coded representation of the results of the genetic test corresponding to each of the wells.

16. The method of claim 14, further comprising the user interface including a patient report associated with each of the patient samples, at least one of the patient reports optionally including a comment associated with the allele call, wherein the comment comprises a predefined comment or a custom comment.

17. The method of claim 16, further comprising a user password operable to provide at least one level of access to the comment in the patient reports.

18. The method of claim 14, further comprising:
a plurality of the patient samples including a similar allele call; and
a plurality of patient reports associated with the plurality of patient samples, each of the plurality of patient reports including a comment describing the similar allele call.

19. The method of claim 14, further comprising filtering the results of the genetic test for each of the patient samples based on the allele call.

20. The method of claim 1, further comprising configuring at least one of:
a range of allele ratios associated with an allele call, the range including at least one cutoff ratio;
at least one equivocal zone surrounding the at least one cutoff ratio, the at least one equivocal zone including an upper boundary and a lower boundary;
a scale factor operable to be used to generate a virtual line blot;
a high signal threshold operable to be used to validate the control bead; and
a low signal threshold operable to be used to validate the control bead.

21. A system for processing results of a complex genetic test, comprising:
a genetic analyzer operable to:
perform a genetic test on a plurality of patient samples contained in a plurality of wells on a plate, the patient samples including at least one control bead operable to provide at least one fluorescent control signal; and
collect raw data for the genetic test; and
a computer system including a processor, a computer readable memory operably coupled to the processor, and processing instructions encoded in the computer readable memory, the processing instructions, when executed by the processor, operable to perform operations comprising:
calculating results of the genetic test by applying a signal intensity associated with the fluorescent control signal to the raw data; and
generating a user interface operable to display the results associated with each of the patient samples.

22. The system of claim 21, further comprising the control bead operable to define at least one of a minimum signal intensity associated with each of the wells, quality control metrics for the genetic test, and quality control metrics associated with the patient sample.

23. The system of claim 21, further comprising the control bead operable indicate whether the genetic test was performed successfully.

24. The system of claim 21, further comprising the patient samples including at least two control beads, each of the control beads operable to provide a unique signal intensity.

25. The system of claim 24, further comprising the control beads including a capture probe covalently bonded to a microsphere, the capture probe operable to hybridize with an exogenous target contained in the patient samples.

26. The system of claim 21, further comprising:
the genetic analyzer operable to perform a reflex test and collect reflex raw data if the results of the genetic test for at least one of the patient samples indicate a specific genetic mutation, the reflex test operable to determine a severity associated with the specific genetic mutation; and
the processing instructions operable to perform operations including:
calculating the results of the reflex test by applying the signal intensity associated with the fluorescent control signal to the reflex raw data for the reflex test; and
integrating the results of the reflex test with the results of the genetic test in the user interface for the at least one of the patient samples.

27. The system of claim 21, further comprising:
the genetic analyzer operable to simultaneously perform the genetic test to collect raw data and a reflex test to collect reflex raw data, the reflex test operable to determine a severity associated with a specific genetic mutation; and
the processing instructions operable to perform operations including:
calculating results of the reflex test by applying the signal intensity associated with the fluorescent control signal to the reflex raw data if the results of the genetic test for at least one of the patient samples indicate the specific genetic mutation; and
integrating the results of the reflex test with the results of the genetic test in the user interface for the at least one of the patient samples.

28. The system of claim 27, further comprising:
the genetic analyzer operable to generate an output file including the raw data for the genetic test, the reflex raw data for the reflex test and the signal intensity associated with the fluorescent control signal; and
the processing instructions operable to perform operations including parsing the output file to generate:
a mutation file including the raw data for the genetic test and the signal intensity associated with the fluorescent control signal; and
a reflex file including the reflex raw data for the reflex test and the signal intensity associated with the fluorescent control signal.

29. The system of claim 21, further comprising the processing instructions operable to perform operations including:
validating a quality associated with the patient samples using the signal intensity; and
calculating the results of the genetic test if the control bead is validated.

30. The system of claim 21, wherein calculating the results of the genetic test comprises calculating background corrected data from the raw data by using the signal intensity associated with the fluorescent control signal.

31. The system of claim 30, further comprising the processing instructions operable to perform operations including generating a virtual line blot generated based on the background corrected data for display in the user interface, the virtual line blot operable to provide a visual representation of the results of the genetic test.

32. The system of claim 30, further comprising the processing instructions operable to perform operations including calculating at least one of an allele ratio or an allele call based on the background corrected data.

33. The system of claim 32, further comprising the processing instructions operable to perform operations including:
generating a summary chart containing at least one of the raw data for the genetic test for display in the user interface, the background corrected data, the allele ratio and the allele call associated with each of the patient samples; and
generating a plate chart including a color coded representation of the results of the genetic test corresponding to each of the wells for display in the user interface.

34. The system of claim 32, further comprising the processing instructions operable to perform operations including generating a patient report associated with each of the patient samples for display in the user interface, at least one of the patient reports optionally including a comment associated with the allele call, wherein the comment comprises a predefined comment or a custom comment.

35. The system of claim 34, further comprising the processing instruction operable to perform operations including accessing a comment history associated with the comment from a database operably coupled to the computer system.

36. The system of claim 32, further comprising the processing instructions operable to perform operations including filtering the results of the genetic test for each of the patient samples based on the allele call.

37. A method for processing results of a complex genetic test, comprising:
receiving raw data from a genetic analyzer operable to perform a genetic test on a plurality of patient samples contained in a plurality of wells on a plate;
calculating results of the genetic test based on the raw data; and
generating a user interface operable to display at least one of:
a summary chart including the raw data and the calculated results; and
a patient report associated with the calculated results for each of the patient samples.

38. The method of claim 37, wherein calculating the results of the genetic test comprises calculating background corrected data from the raw data.

39. The method of claim 38, further comprising the user interface operable to display a virtual line blot generated based on the background corrected data and a scaling factor, the virtual line blot operable to provide a visual representation of the results of the genetic test.

40. The method of claim 38, further comprising calculating at least one of an allele ratio or an allele call based on the background corrected data.

41. The method of claim 40, further comprising filtering the results of the genetic test for each of the patient samples based on the allele call.

42. The method of claim 40, further comprising at least one of the patient reports optionally including a comment associated with the allele call, wherein the comment comprises predefined comment or a custom comment.

43. The method of claim 42, further comprising a user password operable to provide at least one level of access to the comment in the patient reports.

44. The method of claim 40, further comprising:
a plurality of the patient samples including a similar allele call; and
a plurality of patient reports associated with the plurality of patient samples, each of the plurality of patient reports including a comment describing the similar allele call.

45. The method of claim 37, further comprising the user interface operable to display a plate view including a color-coded representation of the results of the genetic test corresponding to each of the wells.

46. The method of claim 37, further comprising configuring at least one of:
a range of allele ratios associated with an allele call, the range including at least one cutoff ratio;
at least one equivocal zone surrounding the at least one cutoff ratio, the at least one equivocal zone including an upper boundary and a lower boundary;
a scale factor operable to be used to generate a virtual line blot;
a high signal threshold operable to be used to validate the control bead; and
a low signal threshold operable to be used to validate the control bead.

47. The method of claim 37, further comprising:
receiving reflex raw data for a reflex test from the genetic analyzer if the results of the genetic test for at least one of the patient samples indicate a specific genetic mutation, the reflex test operable to determine a severity associated with the specific genetic mutation;
calculating results of the reflex test based on the reflex raw data; and
integrating the results of the reflex test with the results of the genetic test in the user interface for the at least one of the patient samples.

48. The method of claim 37, further comprising:
simultaneously receiving the raw data for the genetic test and reflex raw data for a reflex test from the genetic analyzer, the reflex test operable to determine a severity associated with a specific genetic mutation;
calculating results of the reflex test based on the reflex raw data if the results of the genetic test for at least one of the patient samples indicate a specific genetic mutation; and
integrating the results of the reflex test with the results of the genetic test in the user interface for the at least one of the patient samples.

49. The method of claim 48, further comprising:
receiving the raw data for genetic test and the reflex raw data for the reflex test in an output file generated by the genetic analyzer; and
parsing the output file to generate a mutation file including the raw data for the genetic test and a reflex file including the reflex raw data for the reflex test.

50. The method of claim 37, further comprising:
the patient samples including at least one control bead operable to provide at least one fluorescent control signal; and
calculating the results of the genetic test by applying a signal intensity associated with the fluorescent control signal to the raw data.

51. The method of claim 50, further comprising the control bead operable to define at least one of a minimum signal intensity associated with each of the wells, quality control metrics for the genetic test, and quality control metrics associated with the patient sample.

52. The method of claim 50, further comprising the control bead operable to facilitate background correction of the raw data to generate background corrected data.

53. A graphical user interface for displaying results of a complex genetic test on a display device, comprising:
a summary chart containing at least one of:
raw data associated with a plurality of patient samples collected by a genetic analyzer operable to perform a genetic test;
background corrected data calculated based on the raw data corresponding to each of the patient samples;
an allele ratio calculated based on the background corrected data corresponding to each of the patient samples; and
an allele call determined based on the allele ratio corresponding to each of the patient samples; and
a patient report associated with each of the patient samples.

54. The interface of claim 53, further comprising a virtual line blot generated based on the background corrected data and a scale factor, the virtual line blot operable to provide a visual representation of the results of the genetic test.

55. The interface of claim 53, further comprising a plate view including a color-coded representation of the results of the genetic test corresponding to each of a plurality of wells containing the patient samples.

56. The interface of claim 53, further comprising a batch select operable to filter the results of the genetic test for each of the patient samples based on the allele call.

57. The interface of claim 53, further comprising at least one of the patient reports optionally including a comment associated with the allele call, wherein the comment comprises predefined comment or a custom comment.

58. The interface of claim 57, further comprising a user password operable to provide a level of access to the comment in the patient report, wherein the level of access includes viewing the comment, adding the comment or modifying the comment.

59. The interface of claim 53, further comprising:
the patient samples including at least one control bead operable to provide at least one fluorescent control signal; and
the control bead operable to facilitate background correction of the raw data to generate the background corrected data.

60. Software for processing results of a complex genetic test, the software being embodied in computer-readable media and when executed operable to:
receive raw data from a genetic analyzer operable to perform a genetic test on a plurality of patient samples contained in a plurality of wells on a plate;
calculate results of the genetic test based on the raw data; and
generate a user interface operable to display at least one of a summary chart including the raw data and the calculated results and a patient report associated with the results for each of the patient samples.

61. The software of claim 60, wherein the results of the genetic test comprise background corrected data calculated from the raw data.

62. The software of claim 61, further comprising the user interface operable to display a virtual line blot generated based on the background corrected data and a scaling factor, the virtual line blot operable to provide a visual representation of the results of the genetic test.

63. The software of claim 61, further operable to calculate at least one of an allele ratio or an allele call based on the background corrected data.

64. The software of claim 63, further operable to filter the results of the genetic test for each of the patient samples based on the allele call.

65. The software of claim 63, further comprising at least one of the patient reports optionally including a comment associated with the allele call, the comment operable to be accessed by a user password.

66. The software of claim 65, further operable to access a comment history associated with the comment from a database.

67. The software of claim 60, further operable to:
receive reflex raw data for a reflex test from the genetic analyzer if the results of the genetic test for at least one of the patient samples indicate a specific genetic mutation, the reflex test operable to determine a severity associated with the specific genetic mutation;
calculate results of the reflex test based on the reflex raw data; and
integrate the results of the reflex test with the results of the genetic test in the user interface for the at least one of the patient samples.

68. The software of claim 60, further operable to:
simultaneously receive the raw data for the genetic test and reflex raw data for a reflex test from the genetic analyzer, the reflex test operable to determine a severity associated with a specific genetic mutation;
calculate results of the reflex test based on the reflex raw data if the results of the genetic test for at least one of the patient samples indicate the specific genetic mutation; and
integrate the results of the reflex test with the results of the genetic test in the user interface for the at least one of the patient samples.

69. The software of claim 68, further operable to:
receive the raw data for genetic test and the reflex raw data for the reflex test in an output file generated by the genetic analyzer; and
parse the output file to generate a mutation file including the raw data for the genetic test and a reflex file including the reflex raw data for the reflex test.

70. The software of claim 60, wherein:
the patient samples comprise at least one control bead operable to provide at least one fluorescent control signal; and
the results of the genetic test are calculated by applying a signal intensity associated with the fluorescent control signal to the raw data.

71. The software of claim 69, further comprising the control bead operable to facilitate background correction of the raw data to generate background corrected data.
